(19) United States
(12) Patent Application Publication
(10) Pub. No.: US 2013/0274824 A1
(43) Pub. Date: Oct. 17, 2013

(54) NEUROMODULATORY METHOD FOR TREATING CHRONIC RHINOSINUSITIS

(71) Applicant: THE OHIO STATE UNIVERSITY, Columbus, OH (US)

(72) Inventors: Bradley A. Otto, Columbus, OH (US); Ali R. Rezai, Columbus, OH (US)

(73) Assignee: The Ohio State University, Columbus, OH (US)

(21) Appl. No.: 13/864,566

(22) Filed: Apr. 17, 2013

Related U.S. Application Data
(60) Provisional application No. 61/625,356, filed on Apr. 17, 2012.

(51) Int. Cl. A61N 1/36; A61M 5/00

(52) U.S. Cl. CPC A61N 1/36135; A61M 5/00
USPC 607/45; 607/118; 604/93.01

(57) ABSTRACT

One aspect of the present invention includes a method for treating chronic rhinosinusitis (CRS) in a subject. One step of the method includes implanting a therapy delivery system in the subject so that at least one therapy delivery component of the system is positioned substantially adjacent a target location where modulation of the autonomic nervous system (ANS) is effective to treat CRS. The therapy delivery component includes at least one electrode configured to deliver electric current to the target location. Next, electric current is delivered to the at least one electrode to effect a change in the ANS.

### RELATED APPLICATION

This application claims the benefit of U.S. Provisional Patent Application Ser. No. 61/625,356, filed Apr. 17, 2012, the entirety of which is hereby incorporated by reference for all purposes.

### TECHNICAL FIELD

The present disclosure relates to a neuromodulatory method for treating inflammation of the nasal passages and sinus cavities, and in particular to a method of treating chronic rhinosinusitis by modulation of the autonomic nervous system.

### BACKGROUND

Broadly defined, rhinosinusitis is an inflammatory condition of the nasal cavity and paranasal sinuses. Traditionally, rhinosinusitis has been broadly classified by symptom duration: acute (less than four weeks); subacute (4-12 weeks); and chronic (greater than 12 weeks). Four or more episodes of acute sinusitis per year with complete symptom resolution between episodes are characterized as recurrent acute rhinosinusitis. Conversely, chronic rhinosinusitis (CRS) is characterized by relatively persistent symptoms, although these patients may experience several acute exacerbations per year.

In general, rhinosinusitis is an incompletely understood and controversial disease process. Although it is presumed, and generally accepted, that acute rhinosinusitis is infectious in nature, objective measurements that distinguish viral versus bacterial infection are generally lacking in most primary care settings. Given that the disease prevalence of all forms of rhinosinusitis in the U.S. population is about 15%, and the fact that one in five prescriptions for antibiotics is for rhinosinusitis, there exists significant risk of antibiotic over-prescription. In contrast to acute rhinosinusitis, there currently exists no consensus on the underlying etiology of CRS.

Despite a prevalence of about 12.5% of the U.S. population, and a disease specific quality-of-life profile similar to chronic heart disease, CRS remains a poorly understood disease. Diagnosis of CRS is clinically based, with both subjective and objective criteria. Symptomatic criteria include nasal obstruction or congestion, nasal drainage, decreased or absent smell, and facial pressure or headache. Objective criteria include computed tomography (CT) scan evidence of CRS or evidence of CRS (edema, pus, or polyps) on nasal endoscopy. Patients who meet diagnostic criteria are generally stratified into one of three main groups: CRS with nasal polyps (CRSwNP); CRS without nasal polyps (CRSsNP); and allergic fungal sinusitis (AFS). Taken together, CRSwNP and CRSsNP account for about 90% of the total CRS population. Although it may seem that CRSsNP is the clinical equivalent of CRSwNP, void of polyps, the disease processes are much more complex—it is likely that each represents an entirely distinct disease process, with separate etiologic factors, that culminate in a similar symptom profile.

Based on initial clinical research demonstrating differences in the clinical response and natural course of CRSwNP and CRSwNP, there has been exponential growth in basic science research aimed at understanding the molecular and microbiologic processes involved in CRS. However, the topic remains controversial, as several entities (such as Staphylococcus aureus enterotoxins, fungus, biofilms, epithelial immune barrier dysfunction, T helper 17 cells [Tₕ17 cells], among others), have been proposed, but not consistently confirmed, as etiological factors. Other research has clearly demonstrated that significant molecular heterogeneity exists not only among CRS subgroups, but also within the CRSwNP and CRSsNP subgroups. For example, the inflammatory profile in polyps from Chinese patients is generally Tₕ1/Tₕ17 polarized with a neutrophilic predominance, while that of Western populations is generally Tₕ2 polarized with an eosinophilic profile. However, even within these populations, this trend is not consistent. Disparity such as this points to the difficulty in pinning down one etiological agent or cascade that leads to all cases of CRS. In fact, CRS is likely a multi-factorial disease with genetic, environmental, locoregional and/or systemic factors acting in concert to affect the subjective and objective changes characteristic of CRS.

As might be suspected, our poor understanding of the pathogenesis of CRS has restricted our ability to effectively treat all patients with CRS. A number of medical regimens have been proposed, incorporating agents such as steroids, antihistamines, antibiotics, and other anti-inflammatory medications. However, effectiveness as demonstrated by randomized, controlled trial is lacking. Accordingly, there currently exists no U.S. Food and Drug Administration approval for any medication aimed at CRS, with the exception of mometasone.

Despite aggressive medical therapy, many patients remain symptomatic from the ongoing inflammatory process. For these patients, surgical intervention is frequently offered as an adjunct to ongoing anti-inflammatory therapy. The goal of most sinus procedures is to restore paranasal sinus ventilation and improve drainage and hence restore normal physiologic function of the sinuses. However, a standardized method of performing sinus surgery is not universally accepted. Although results vary, most sinus surgery is effective in reducing symptoms. However, up to 20% of patients will require revision surgery due to ongoing symptoms. Furthermore, many patients, even if improved, continue to have symptoms despite ongoing medical therapy after surgery. For these recalcitrant patients, there currently exists little to offer other than revision surgery and ongoing medications.

### SUMMARY

One aspect of the present disclosure includes a method for treating chronic rhinosinusitis (CRS) in a subject. One step of the method includes implanting a therapy delivery system in the subject so that at least one therapy delivery component of the system is positioned substantially adjacent a target location where modulation of the autonomic nervous system (ANS) is effective to treat CRS. The therapy delivery component includes at least one electrode configured to deliver electric current to the target location. Next, electric current is delivered to the at least one electrode to effect a change in the ANS.

Another aspect of the present disclosure includes a method for treating CRS in a subject. One step of the method includes implanting a closed-loop therapy delivery system in the subject so that at least one therapy delivery component of the system is positioned substantially adjacent a target location where modulation of the ANS is effective to treat CRS. The therapy delivery component includes at least one electrode configured to deliver electric current to the target location. Next, electric current is delivered to the at least one electrode to effect a change in the ANS. At least one physiological parameter associated with CRS is then sensed by the therapy delivery system. The therapy delivery component of the system is activated to adjust application of the electric current to the target site in response to the sensed at least one physiological parameter.

### BRIEF DESCRIPTION OF THE DRAWINGS

The foregoing and other features of the present disclosure will become apparent to those skilled in the art to which the present disclosure relates upon reading the following description with reference to the accompanying drawings, in which:

FIG. 1 is a process flow diagram illustrating a method for treating chronic rhinosinusitis (CRS) according to one aspect of the present disclosure;

FIG. 2 is a schematic drawing of a lateral view of the skull showing the position of the infratemporal fossa with the sphenopalatine ganglion (SPG) lying within the sphenopalatine fossa, surrounded by the anterior margin of the lateral pterygoid plate and the posterior wall of the maxillary sinus;

FIG. 3 is a schematic illustration of a lateral view of the lateral nasal wall showing the position of the SPG directly underneath the nasal mucosa and located at the posterior margin of the superior and middle nasal turbinates;

FIG. 4 is a schematic view of anatomical tissue structures including the maxillary, frontal, and ethmoid sinus cavities;

FIG. 5A is a frontal view of a human head showing the locations of the paranasal sinuses;

FIG. 5B is a side view of a human head showing the locations of the paranasal sinuses;

FIG. 6 is a process flow diagram illustrating a method for treating CRS according to another aspect of the present disclosure; and

FIG. 7 is a schematic illustration showing an implanted closed-loop therapy delivery system according to another aspect of the present disclosure.

### DETAILED DESCRIPTION

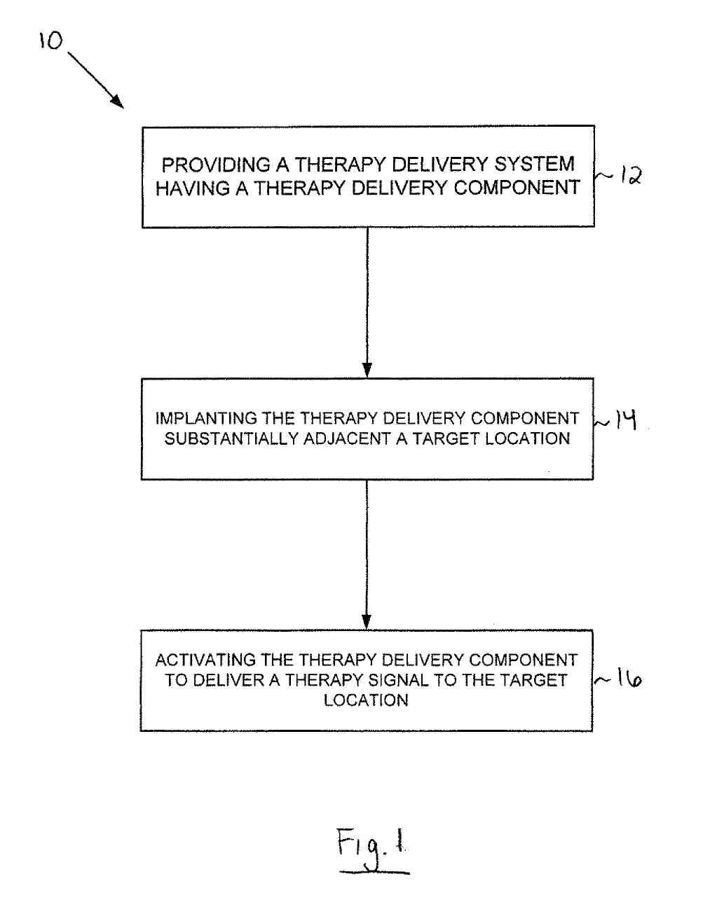

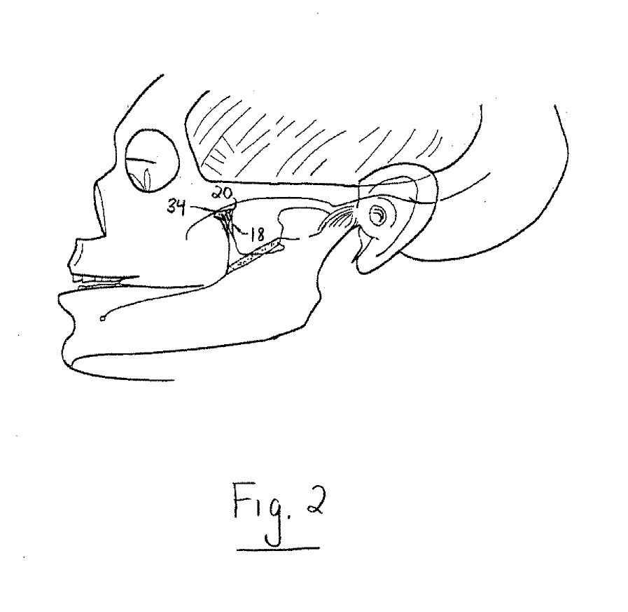

The present disclosure relates to a neuromodulatory method for treating inflammation of the nasal cavity and paranasal sinuses, and in particular to a method of treating chronic rhinosinusitis (CRS) by modulation of the autonomic nervous system (ANS). As representative of one aspect of the present disclosure, FIG. 1 illustrates a method 10 for treating CRS in a subject. The present disclosure addresses physiologic changes and symptoms associated with CRS by focusing treatment on the ANS and, in particular, nerve structures or nervous tissue associated with the pterygopalatine fossa (PPF) 18 (FIG. 2) to modulate the neurovascular contribution to sinonasal immunity and physiology. As described in more detail below, the present disclosure can deliver therapy either on-demand or continuously in a dynamic fashion. Consequently, therapy can be titrated based on real-time conditions and symptoms associated with CRS.

Unless otherwise defined, all technical terms used herein have the same meaning as commonly understood by one of ordinary skill in the art to which the present disclosure pertains.

In the context of the present disclosure, the terms “nervous tissue” and “nerve structure” can refer to any tissues of the ANS including, but not limited to, neurons, axons, fibers, tracts, nerves, plexus, afferent plexus fibers, efferent plexus fibers, ganglion, pre-ganglionic fibers, post-ganglionic fibers, cervical sympathetic ganglia/ganglion, thoracic sympathetic ganglia/ganglion, afferents, efferents, and combinations thereof.

As used herein, the terms “modulate” or “modulating” can refer to causing a change in neuronal activity, chemistry, and/or metabolism. The change can refer to an increase, decrease, or even a change in a pattern of neuronal activity. The terms may refer to either excitatory or inhibitory stimulation, or a combination thereof, and may be at least electrical, magnetic, optical or chemical, or a combination of two or more of these. The terms “modulate” or “modulating” can also be used to refer to a masking, altering, overriding, or restoring of neuronal activity.

As used herein, the terms “chronic rhinosinusitis” or “CRS” can refer to the disease entity characterized by inflammation of the nasal cavity and/or paranasal sinuses lasting greater than about twelve weeks duration. Symptoms may include, but are not limited to: facial pain or pressure; nasal congestion or fullness; nasal obstruction or blockage; nasal discharge (rhinorrhea or post-nasal drip); hyposmia/anosmia; and purulence in the nasal cavity. Other potential symptoms include: headache; fever; halitosis; fatigue; dental pain; cough; and ear pain/pressure/fullness. In one example, the presence of severe inflammation and irritation with thickened discolored or purulent discharge can be indicative of CRS, whereas pale mucosa with clear discharge can be suggestive of allergic rhinitis. In another example, CRS can refer to recalcitrant forms of the disease in which symptoms persist despite medical or surgical treatment, as well as instances where patients cannot receive standard medical or surgical care due to contraindications for such care.

As used herein, the term “target location” can refer to a suitable anatomical location at which a therapy delivery system, and in particular a therapy delivery component of the system, may be positioned to effect a chance in the ANS. In some instances, the target location can comprise an anatomical location that is innervated by, or in electrical communication with, one or more autonomic and/or sensory nerves extending, or involved in the interplay, between the sinonasal cavity and the PPF. In other instances, the target location can comprise a variety of anatomical locations, including intraluminal and extraluminal locations innervated by, or in electrical communication with, a nerve structure or nervous tissue associated with the PPF, such as a nerve structure or nervous tissue of the ANS. Target locations and associated nerve structures or nervous tissue contemplated by the present disclosure are described in further detail below.

As used herein, the term “electrical communication” can refer to the ability of an electric field generated by an electrode (or electrode array) to be transferred, or to have a neuromodulatory effect, within and/or on at least one nerve, neuron, nerve structure and/or nervous tissue of the ANS.

As used herein, the term “subject” can refer to any warm-blooded organism including, but not limited to, human beings, pigs, rats, mice, dogs, goats, sheep, horses, monkeys, apes, rabbits, cattle, etc.

As used herein, the terms “treating” and “treat” can refer to therapeutically regulating, preventing, improving, alleviating the symptoms of, and/or reducing the effects or symptoms of CRS. The terms can also refer to chronic or acute treatment.

As used herein, the term “therapy signal” can refer to an electrical and/or chemical signal that is delivered to a target location and is capable of modulating (e.g., electrically modulating) a nerve structure or nervous tissue to effect a change in the ANS.

When an element or structure is referred to as being “on,” “engaged to,” “connected to,” or “coupled to” another element or structure, it may be directly on, engaged, connected or coupled to the other element or structure, or intervening elements or structures may be present. In contrast, when an element is referred to as being “directly on,” “directly engaged to,” “directly connected to,” or “directly coupled to” another element or structure, there may be no intervening elements or structures present. Other words used to describe the relationship between elements should be interpreted in a like fashion (e.g., “between” versus “directly between,” “adjacent” versus “directly adjacent,” etc.).

A brief discussion of the neurophysiology is provided to assist the reader with understanding the present disclosure. The nervous system is divided into the somatic nervous system and the ANS. In general, the somatic nervous system controls organs under voluntary control (e.g., skeletal muscles) and the ANS controls individual organ function and homeostasis. For the most part, the ANS is not subject to voluntary control. The ANS is also referred to as the visceral or automatic system.

The ANS can be viewed as a “real-time” regulator of physiological functions that extracts features from the environment and, based on that information, allocates an organism's internal resources to perform physiological functions for the benefit of the organism, e.g., responds to environment conditions in a manner that is advantageous to the organism.

The ANS conveys sensory impulses to and from the central nervous system to various structures of the body such as organs and blood vessels, in addition to conveying sensory impulses through reflex arcs. For example, the ANS controls: constriction and dilatation of blood vessels; heart rate; the force of contraction of the heart; contraction and relaxation of smooth muscle in various organs; lungs; stomach; colon; bladder; and visual accommodation, secretions from exocrine and endocrine glands, etc. The ANS does this through a series of nerve fibers, and more specifically through efferent and afferent nerves. The ANS acts through a balance of its two components: the sympathetic nervous system (SNS) and the parasympathetic nervous system (PNS), which are two anatomically and functionally distinct systems. Both of these systems include myelinated preganglionic fibers, which make synaptic connections with unmyelinated postganglionic fibers, and it is these fibers which then innervate the effector structure. These synapses usually occur in clusters called ganglia. Most organs are innervated by fibers from both divisions of the ANS, and the influence is usually opposing (e.g., the vagus nerve slows the heart, while the sympathetic nerves increase its rate and contractility), although it may be parallel (e.g., as in the case of the salivary glands). Each of these is briefly reviewed below.

The PNS is the part of the ANS controlling a variety of autonomic functions including, but not limited to, involuntary muscular movement of blood vessels and gut and glandular secretions from eye, salivary glands, bladder, rectum and genital organs. The vagus nerve is part of the PNS. Parasympathetic nerve fibers are contained within the last five cranial nerves and the last three spinal nerves and terminate at parasympathetic ganglia near or in the organ they supply. The actions of the PNS are broadly antagonistic to those of the SNS; lowering blood pressure, slowing heartbeat, stimulating the process of digestion etc. The chief neurotransmitter in the PNS is acetylcholine. Neurons of the parasympathetic nervous system emerge from the brainstem as part of the Cranial nerves III, VII, IX and X (vagus nerve) and also from the sacral region of the spinal cord via Sacral nerves. Because of these origins, the PNS is often referred to as the “craniosacral outflow”.

In the PNS, both pre- and post-ganglionic neurons are cholinergic (i.e., they utilize the neurotransmitter acetylcholine). Unlike adrenaline and noradrenaline, which the body takes around 90 minutes to metabolize, acetylcholine is rapidly broken down after release by the enzyme cholinesterase. As a result the effects are relatively brief in comparison to the SNS.

Each pre-ganglionic parasympathetic neuron synapses with just a few post-ganglionic neurons, which are located near, or in, the effector organ, a muscle or gland. As noted above, the primary neurotransmitter in the PNS is acetylcholine such that acetylcholine is the neurotransmitter at all the pre-ganglionic neurons and many of the post-ganglionic neurons of the PNS. Some of the post-ganglionic neurons, however, release nitric oxide as their neurotransmitter.

The SNS is the part of the ANS comprising nerve fibers that leave the spinal cord in the thoracic and lumbar regions and supply viscera and blood vessels by way of a chain of sympathetic ganglia running on each side of the spinal column, which communicate with the central nervous system via a branch to a corresponding spinal nerve. The SNS controls a variety of autonomic functions including, but not limited to, control of movement and secretions from viscera and monitoring their physiological state, stimulation of the sympathetic system inducing, e.g., the contraction of gut sphincters, heart muscle and the muscle of artery walls, and the relaxation of gut smooth muscle and the circular muscles of the iris. The chief neurotransmitter in the SNS is adrenaline, which is liberated in the heart, visceral muscle, glands and internal vessels, with acetylcholine acting as a neurotransmitter at ganglionic synapses and at sympathetic terminals in skin and skeletal muscles. The actions of the SNS tend to be antagonistic to those of the PNS.

The neurotransmitter released by the post-ganglionic neurons is noradrenaline (also called norepinephrine). The action of noradrenaline on a particular structure, such as a gland or muscle, is excitatory in some cases and inhibitory in others. At excitatory terminals, ATP may be released along with noradrenaline. Activation of the SNS may be characterized as general because a single pre-ganglionic neuron usually synapses with many post-ganglionic neurons, and the release of adrenaline from the adrenal medulla into the blood ensures that all the cells of the body will be exposed to sympathetic stimulation even if no post-ganglionic neurons reach them directly.

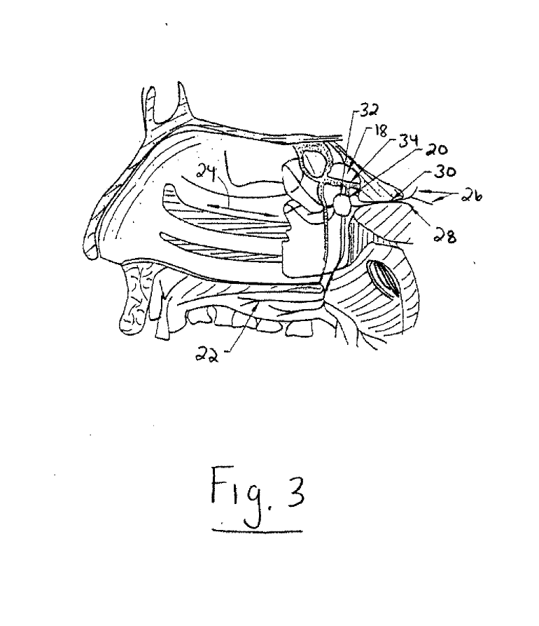

Referring to FIGS. 2-3, the sphenopalatine ganglion (SPG) 20 is located behind the maxilla 22 in the PPF 18 posterior to the middle nasal turbinate 24. The SPG 20 is surrounded by a layer of mucosal and connective tissue of less than five millimeters in thickness. The SPG 20 is part of the parasympathetic division of the ANS; however, the SPG has both sympathetic and parasympathetic nerve fibers, as well as sensory and visceral nerve fibers. The parasympathetic activity of the SPG 20 is mediated through the greater petrosal nerve 26, while the sympathetic activity of the SPG is mediated through the deep petrosal nerve 26, which is essentially an extension of the cervical sympathetic chain. Facial nerve and carotid plexuses directly communicate sensory signals to the SPG 20, and cell bodies in the ventral horn of the thoracolumbar spinal cord send fibers either directly or via cervical ganglion to the SPG.

The deep and greater petrosal nerves 26 join together just before entering the pterygoid canal to form the vidian nerve 28. The vidian nerve 28 is housed within the vidian canal 30, which is directly posterior to the SPG 20. The vidian nerve 28 connects to the SPG 20 and contains parasympathetic fibers, which synapse in the SPG, sensory fibers that provide sensation to part of the nasal septum, and also sympathetic fibers.

The sphenopalatine nerves 32 are sensory nerves that connect the SPG 20 to the maxillary nerve 34. The sphenopalatine nerves 32 traverse through the SPG 20 without synapsing and proceed to provide sensation to the palate. The sphenopalatine nerves 32 suspend the SPG 20 in the PPF.

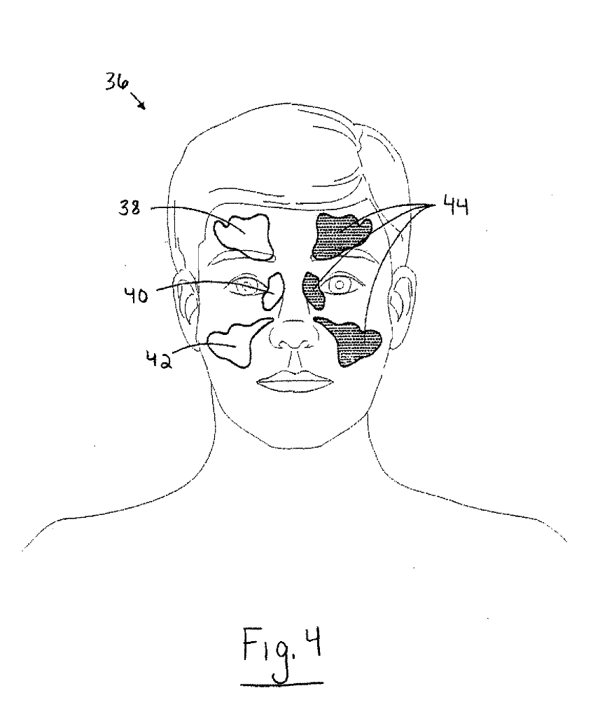

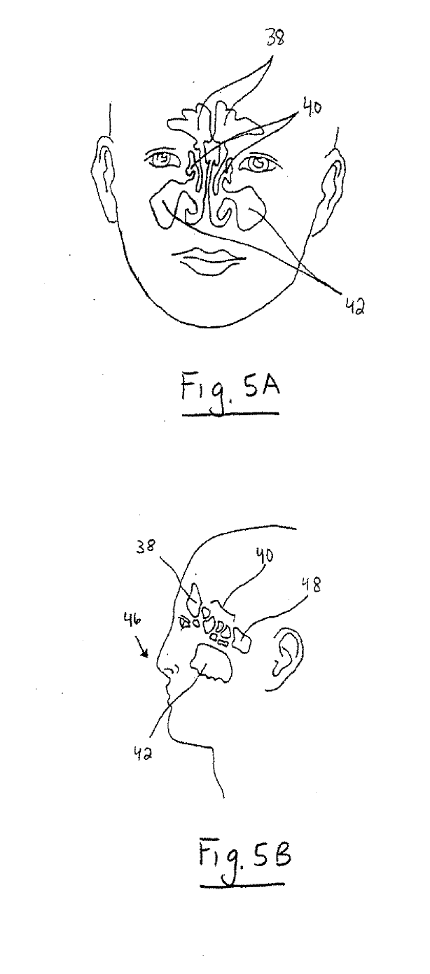

FIGS. 4-5B illustrate anatomical tissue structures 36 associated with sinusitis. There are four different pairs of sinuses: the frontal sinuses 38; the ethmoid sinuses 40; the maxillary sinuses 42; and the sphenoid sinuses (located more toward the back of the head than the other sinuses). Normally, sinuses are filled with air, but when sinuses become blocked and filled with fluid, pathogens can grow and cause an infection. The factors that contribute to the sinus initially becoming blocked off are likely multi-factorial, but may be due to infections, inflammation, allergy, immunologic abnormalities, or other processes. In FIG. 4, the sinuses 44 on the (reader's) right side are shown as inflamed and experiencing CRS.

The human nose 46 (FIGS. 5A-B) has right and left nostrils or nares that lead into separate right and left nasal cavities. The right and left nasal cavities are separated by the intranasal septum, which is formed substantially of cartilage and bone. Posterior to the intranasal septum, the nasal cavities converge into a single nasopharyngeal cavity. The right and left Eustachian tubes (i.e., auditory tubes) extend from the middle ear on each side of the head to openings located on the lateral aspects of the nasopharynx. The nasopharynx extends inferiorly over the uvula and into the pharynx. Paranasal sinuses are formed in the facial bones on either side of the face. The paranasal sinuses open, through individual openings or ostia, into the nasal cavities. As noted above, the paranasal sinuses include frontal sinuses 38, ethmoid sinuses 40, sphenoidal sinuses 48, and maxillary sinuses 42.

Having described the relevant physiology and anatomy to which the present disclosure pertains, one aspect of the present disclosure can include a method 10 for treating CRS in a subject. Referring to FIG. 1, the method 10 can include providing a therapy delivery system 50 having a therapy delivery component 52 (Step 12). At Step 14, the therapy delivery system 50 can be implanted in the subject so that at least one therapy delivery component 52 of the system is positioned substantially adjacent a target location where modulation of the ANS is effective to treat CRS. As described in more detail below, the therapy delivery component 52 can include at least one electrode (not shown) configured to deliver a therapy signal, such as electric current to the target location. After appropriately positioning the therapy delivery component 52, the therapy signal can be delivered to the at least one electrode to effect a change in the ANS and thereby treat the CRS (Step 16).

If left untreated, CRS can lead to serious complications. For example, complications of untreated CRS can include persistent chronic airway obstruction, obstructive sleep apnea and snoring, exacerbation of lower respiratory problems (including asthma), anterior headache, diminished sense of smell and taste, orbital problems (including orbital absceses), meningitis, mucocele formation, fatigue and lower quality of life. Thus, one aspect of the present disclosure can include identifying a subject with CRS. One skilled in the art will appreciate how to identify or diagnose a subject with CRS. Generally, identification of a subject with CRS can include examination of the nasal vault, which can include examining the following: the quality of mucous secretions (e.g., amount, location and thickness); and the presence of purulence, blood or discoloration. Examination can also include examining the nasal mucosa for edema, polyps, inflammation, ulceration or excoriation, erosion, dryness (frequently found in winter months) or atrophy.

In one example, the subject can have a recalcitrant form of CRS in which symptoms persist despite medical or surgical treatment. In another example, the subject treatable by the present disclosure may have CRS but be unable to receive standard medical or surgical care due to contraindications for such care.

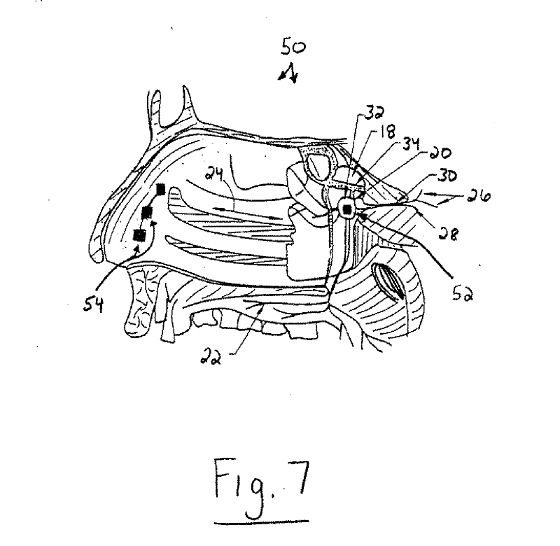

After identifying a subject suffering from CRS, another aspect of the present disclosure can include providing a therapy delivery system 50 (Step 12). The therapy delivery system 50 (FIG. 7), which is not shown in detail, can comprise any medical device, apparatus, or combination thereof configured to deliver a therapy signal to a nerve structure or nervous tissue of the ANS. The therapy delivery system 50 can include at least one electrode that is in electrical communication with a power source (not shown). The power source can include a battery or generator, such as a pulse generator operatively connected to an electrode. Alternatively, power may be supplied to the therapy delivery system 50 via biological energy harvesting. The power source may be positioned in any suitable location, such as integrated as part of the therapy delivery system, adjacent an electrode, at a remote site in or on the subject's body, and/or away from the subject's body in a remote location. One type of power source can include an implantable generator, which may be analogous to a cardiac pacemaker. In one example, one or more electrodes of the therapy delivery system 50 can be indirectly (e.g., wirelessly) connected to the power source.

In some instances, the therapy delivery system 50 can include a drug port (not shown) or other fluid conveying mechanism for delivering at least one pharmacological agent and/or biological agent to the target location. The drug port or other fluid conveying mechanism can be fluidly connected to a reservoir (not shown), which may be implanted within or located remotely from the subject. Any one or combination of pharmacological and/or biological agents can be deliverable to the target location. In some instances, the pharmacological and/or biological agent can include an agent, molecule, cell, compound, or the like capable of modulating ANS activity. In other instances, the pharmacological and/or biological agent can include an agent, molecule, cell, compound, or the like capable of preventing or treating a microbial infection (e.g., an anti-inflammatory agent). In other instances, the pharmacological agent and/or biological agent can be linked to a surface of the therapy delivery system 50 (e.g., one or more surfaces of an electrode), embedded and released from within polymer materials, such as a polymer matrix, or surrounded by and released through a carrier.

In one example, the therapy delivery system 50 can include an implantable neurostimulator. In some instances, the neurostimulator can include a controller (not shown) operably connected to an electrical lead (not shown) having at least one electrode (not shown) connected thereto. The electrode(s) comprising the neurostimulator can be monopolar, bipolar, or multipolar, and can operate as a cathode or an anode. The electrode(s) can be comprised of one or more electrically conductive materials, such as activated iridium, rhodium, titanium, platinum, or a combination thereof. All or only a portion of the electrode(s) may be coated with a thin surface layer of iridium oxide, titanium nitride, or other surface modifications to enhance electrical sensitivity.

The electrical lead can comprise carbon, doped silicon, or silicon nitride. The electrical lead can also be provided with a biocompatible fabric collar or band (not shown) about the periphery of the electrode(s) to allow the electrical lead to be more readily sutured or glued into place. Additionally, the controller can include a fixation plate (e.g., made of titanium) that uses standard anterior craniofacial screws to permit attachment of the neurostimulator to a bony structure (or structures) surrounding the PPF 18, for example.

The controller can be used to operate and/or supply power to the electrode(s). The controller may be powered by the power source. Where the therapy delivery system 50 includes a stimulation lead, the controller may change power output to the electrode(s) by way of polarity, pulse width, amplitude, frequency, voltage, current, and/or waveform. Where the therapy delivery system 50 comprises a drug port, the controller may change its output such that a pump, pressure source, or proportionally controlled orifice increases or decreases the rate at which a pharmacological and/or biological agent is/are delivered to the target location.

The controller may operate any number or combination of electrodes and/or fluid delivery mechanism. For example, the controller may be connected to stimulation leads and a peristaltic pump for delivering electric current and a pharmacological and/or biological agent to the target location. The controller may be entirely implanted within the subject or, alternatively, positioned externally about the subject (e.g., by leads).

Where the controller enables delivery of a electric current to the target location, the electric current may be episodic, continuous, phasic, in clusters, intermittent, upon demand by the subject or medical personnel, or pre-programmed to respond to a sensor (not shown) (e.g., a closed-loop system). The electrical signal can be operated at a constant voltage (e.g., at about 0.1 v to about 25 v), at a constant current (e.g., at about 0.1 microamps to about 5 milliamps), at a constant frequency (e.g., at about 1 Hz to about 10,000 Hz), and at a constant pulse-width (e.g., at about 10 μsec to about 2,000 μsec). Application of electric current can be monopolar, bipolar, or multipolar, depending upon the polarity of the electrode(s). The waveform(s) may be biphasic, square wave, sine wave, or other electrically safe and feasible combinations.

Where the controller enables delivery of a pharmacological and/or biological agent to the target location, the pharmacological and/or biological agent(s) may be delivered to the target location prior to, concurrent with, subsequent to, or instead of electric current. The pharmacological and/or biological agent(s) may be a neurotransmitter mimetic, neuropeptide, hormone, pro-hormone, antagonist, agonist, reuptake inhibitor or degrading enzyme thereof, peptide, protein, chemical agent, amino acid, nucleic acid, stem cell, or any combination thereof, and may be delivered by a slow release matrix or drug pump. Delivery of the pharmacological and/or biological agent(s) may be continuous, intermittent, chronic, phasic or episodic.

The therapy delivery system 50 can be part of an open-loop or closed-loop system. In an open-loop system, for example, a physician or subject may, at any time, manually or by the use of pumps, motorized elements, etc. adjust treatment parameters, such as pulse amplitude, pulse width, pulse frequency, or duty cycle of an electric current. Thus, in an open-loop system, therapy can be delivered on-demand. Alternatively, in a closed-loop system, treatment parameters (e.g., electric current) may be automatically (e.g., continuously) adjusted in response to a sensed physiological parameter (e.g., a symptom) or a related physiological parameter indicative of the extent of the CRS being treated. In a closed-loop feedback system, one or more sensors 54 (FIG. 7) configured to detect at least one physiological parameter associated with CRS can be utilized. More detailed descriptions of sensors 54 that may be employed in a closed-loop system, as well as other examples of sensors and feedback control techniques that may be employed are disclosed in U.S. Pat. No. 5,716,377, which is hereby incorporated by reference in its entirety.

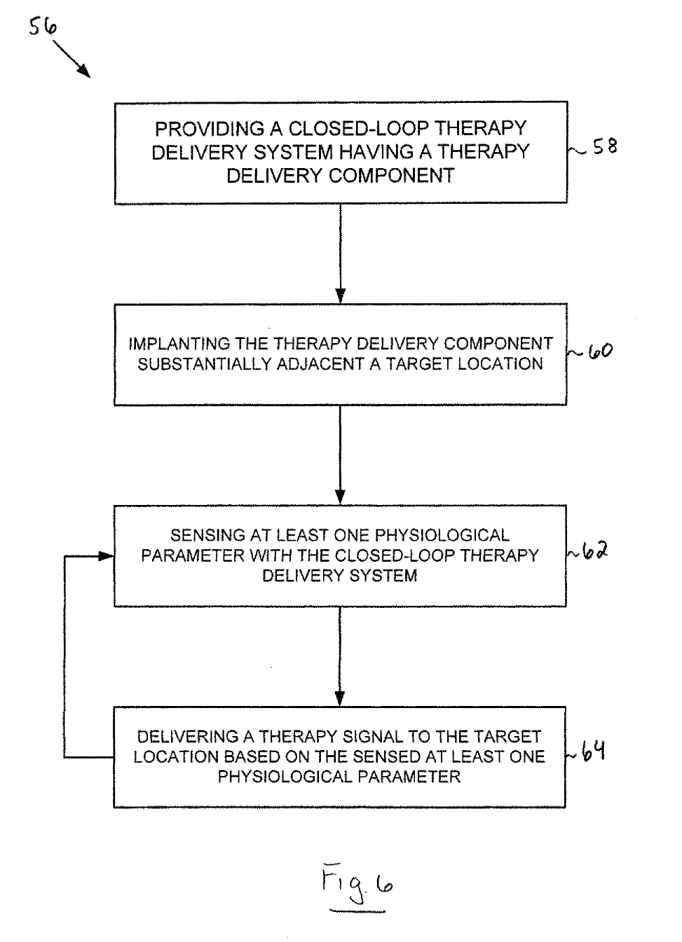

Although described in more detail below, it should be appreciated that incorporating the therapy delivery system 50 as part of a closed-loop method 56 for treating CRS can include the following steps (FIG. 6): providing a closed-loop therapy delivery system 50 having a therapy delivery component 52 (Step 58); implanting the therapy delivery component substantially a target location (Step 60); sensing at least one physiological parameter (Step 62); and delivering a therapy signal to the target location based on the sensed physiological parameter(s) (Step 64).

Physiological parameters detectable by the method 56 can include any characteristic, symptom, molecule, or function of the body that is associated with CRS. Examples of such physiological parameters can include, but are not limited to, mucosal blood flow, sinusoidal filling, mucosal thickness, mucosal secretion, protein or chemical concentrations (e.g., cytokines, histamines), pressure (e.g., sinonasal intraluminal pressure), temperature, pH, mucosal thickness, changes related to mucosal remodeling (e.g., basement membrane thickness, epithelial damage), electrochemical gradients, microbial products and byproducts, gases (e.g., nitric oxide), as well as other locoregional or systemic conditions or changes, such as other gross or molecular changes characteristic of CRS. Example of such molecular changes can include up-regulation and/or down-regulation of various proteins (or their receptors). Such proteins may include, but are not limited to: interferon-alpha; interferon-gamma; interleukins (IL), such as IL-1-beta, IL-2, IL3-, IL-4, IL-5, IL-6, IL-7, IL-8, IL-10, IL-13, IL-15 and IL-17; growth-related oncogene-alpha; epithelial cell-derived neutrophil attractant-78; granulocyte chemotactic protein-2; eotaxin; released upon activation T-cell secreted (RANTES); thymus and activation-regulated chemokine (TARC), matrix metalloproteinases; vascular cell adhesion molecule-1; tumor necrosis factor-alpha; transforming growth factor-beta; chemokines (such as CCL13, CCL2, CCL8, CCL11, CCL18, CCL22, CXCL13); immunoglobulins; toll-like receptors; G-CSF; GM-CSF; MIP-1; VEGF; EGF; HGF; or other protein markers of inflammation.

In addition, the inflammatory cell profile of the sinonasal mucosa may be monitored. For instance, relative or absolute eosinophil, neutrophil, macrophage, or lymphocyte (Th1, Th2, Th17) counts may be determined. Alterations in dendritic cells or associated proteins may also be used. Other markers may include nitric oxide and/or its synthases or metabolites, oxygen tension, and markers of ciliary dysfunction or defects in mucociliary flow. Microbes and/or their byproducts may also be used as markers. For example, bacteria such Staphylococcus aureus or Pseudomonas aeruginosa, fungi, or viruses as well as by-products of these or other organisms may be used. Additionally, gene transcripts, protein markers, or markers of microbial biofilms may be used.

Another aspect of the present disclosure can include implanting the therapy delivery system 50 in the subject so that at least one therapy delivery component 52 (e.g., an electrode) is positioned substantially adjacent the target location and is in electrical communication with one or more nerve structure(s) or nervous tissue(s) associated with the PPF 18. In certain aspects, the target location can include at least one of the SPG 20, a greater palatine nerve (not shown in detail), a lesser palatine nerve (not shown in detail), a sphenopalatine nerve 32, a communicating branch between a maxillary nerve 34 and an SPG, an otic ganglion (not shown), an afferent fiber going into the otic ganglion, an efferent fiber going out of the otic ganglion, an infraorbital nerve (not shown), a vidian nerve 28, a greater superficial petrosal nerve 26, a lesser deep petrosal nerve 26, a trigeminal nerve (not shown), a posterior inferior lateral nasal branch (not shown) of the maxillary nerve, a anterior superior alveolar nerve (not shown), a nasopalatine nerve (not shown), an infraorbital nerve (not shown), a posterior superior alveolar nerve (not shown), and an anterior ethmoidal nerve and its branches (e.g., a medial internal nasal branch, and a lateral internal nasal branch, an external nasal branch), as well as any other nerve, branch, or tributary of the other nerves comprising the ANS (discussed above).

In some instances, the therapy delivery component 52 is placed within the PPF 18 or, more specifically, in very close proximity to a nerve structure or nervous tissue associated with the PPF. In one example, the nerve structure or nervous tissue includes at least one of the SPG 20, the vidian nerve 28, or the sphenopalatine nerves 32. In other instances, the nerve structure or nervous tissue includes the SPG 20.

The therapy delivery system 50, and in particular the therapy delivery component 52, can be delivered to and implanted at the target location via any one or combination of surgical approaches. In some instances, the therapy delivery system 50 can be implanted substantially adjacent a target location where modulation of the SNS is effective to treat CRS. In other instances, the therapy delivery system 50 can be implanted substantially adjacent a target location where modulation of the PNS is effective to treat CRS. In other aspects, the therapy delivery system 50 can be implanted substantially adjacent a target location where modulation of the SNS and the PNS is effective to treat CRS.

In other aspects, the therapy delivery system 50 can be delivered to the target location through the greater palatine canal via a trans-palatal approach as disclosed in U.S. Patent Publication No. 2010/0049230 A1 to Benary et al., the entirety of which is hereby incorporated by reference. In other instances, the therapy delivery system 50 can be delivered to the target location via a trans-nasal approach as disclosed in U.S. Patent Publication No. 2006/0195169 A1 to Gross et al., the entirety of which is hereby incorporated by reference. In another aspect, the therapy delivery system 50 can be delivered to the target location via a trans-coronoid notch approach as disclosed in U.S. Pat. No. 6,526,318 to Ansarinia, the entirety of which is hereby incorporated by reference. In other aspects, the therapy delivery system 50 can be delivered to the target location via a gingivo-buccal approach as disclosed in U.S. Patent Publication No. 2010/0185258 A1 to Papay, the entirety of which is hereby incorporated by reference. It will be appreciated that any of the foregoing surgical procedures, as well as any other suitable percutaneous, laparoscopic, or open surgical procedure may also be used to implant the therapy delivery system 50.

Where the therapy delivery system 50 is part of a closed-loop system, one or more sensors 54 can be placed or implanted in the subject to allow detection of at least one physiological parameter associated with CRS. In some instances, one or more sensors 54 can be implanted in the nasal passage or nasal cavity of the subject. For example, one or more sensors 54 can be securely affixed to the mucous membrane lining a portion of the nasal passage or nasal cavity. In other instances, one or more sensors 54 can be securely affixed within one or more of the paranasal sinuses, such as the frontal sinuses 38, the ethmoid sinuses 40, the sphenoidal sinuses 48, and the maxillary sinuses 42.

The sensor(s) 54 can be arranged in any suitable configuration. In some instances, only a single sensor 54 can be implanted. In other instances, two or more sensors 54 can be implanted. In one example, a sensor array comprising three sensors 54 can be implanted in the nasal passage of the subject (FIG. 7). Where a sensor array is used, different sensors 54 can detect different physiological parameters. Alternatively, two or more sensors 54 comprising a sensor array can each detect the same physiological parameter, albeit at a different concentration. A sensor array can be configured in series, as shown in FIG. 7, or in any other configuration to facilitate detection of one or more physiological parameters associated with CRS.

In another aspect, the therapy delivery system 50 (e.g., the therapy delivery component 52) can be activated to deliver a therapy signal (e.g., electric current) to the target location following implantation of the therapy delivery system. The ANS controls blood supply into the nasal mucosa and the secretion of mucus. The diameter of the resistance vessels in the nose 46 is mediated by the SNS, while the PNS controls glandular secretion and, to a lesser extent, exerts an effect on the capacitance vessels. Either a hypoactive SNS or a hyperactive PNS can engorge these vessels, creating an increased swelling of the nasal mucosa, and thus congestion. Additionally, activation of the PNS can increase mucosal secretions leading to excess runny nose.

In one aspect, a therapy signal (e.g., electric current) can be delivered to a target location, such as the SPG 20 to effectively block or reduce parasympathetic activity. Blocking or reduction of parasympathetic activity can decrease or alleviate at least one symptom associated with CRS, such as swelling of nasal mucosa and mucosal secretion. For example, blocking or reduction of parasympathetic activity can cause decreased swelling of the nasal mucosa, which results in clearance of the orifices of the nasal sinuses and decreased blockage, thereby promoting normal drainage. It will be appreciated that delivery of a therapy signal to a parasympathetic nerve structure can block or inhibit efferent and/or afferent neuronal activity thereof as well.

In another aspect, a therapy signal (e.g., electric current) can be delivered (e.g., selectively delivered) to a target location, such as the sympathetic fibers comprising the SPG 20 and/or the vidian nerve 28 to substantially activate or increase sympathetic activity. Activating or increasing sympathetic activity can decrease or alleviate at least one symptom associated with CRS, such as mucosal blood flow, sinusoidal filling, and mucosal thickness. For example, activating or increasing sympathetic activity can promote arterial vasoconstriction, thereby reducing mucosal blood flow, sinusoidal filling, and mucosal thickness, in addition to restoring nasal patency. It will be appreciated that delivery of a therapy signal to a sympathetic nerve structure can increase or activate efferent and/or afferent neuronal activity thereof as well. In other instances, it will be appreciated that one or more therapy signals can be concurrently or intermittently delivered to both sympathetic and parasympathetic nerve structures. For example, a first therapy signal can be delivered to a sympathetic nerve structure to activate or increase sympathetic activity, while a second therapy signal can be delivered to a parasympathetic nerve structure to decrease or block parasympathetic activity.

In a further aspect (FIGS. 6-7), a therapy signal (e.g., electric current) can be delivered to a target location, such as the SPG 20 (as described above). In a closed-loop configuration, one or more sensors 54 located in the nasal cavity and/or paranasal sinus(es) of the subject can then detect at least one physiological parameter associated with CRS, such as mucosal blood flow or mucosal secretion. The therapy delivery component 52 (e.g., an electrode) can be activated to deliver a therapy signal, such as electric current to the target location. Where electric current is delivered to the SPG 20 (e.g., to block or decrease parasympathetic activity), the amount or volume of nasal secretions may decrease as a result. The implanted sensor(s) 54 can then detect the decreased amount or volume of nasal secretions. Based on the detected amount or volume of nasal secretions, the therapy delivery system 50 can adjust the therapy signal accordingly. For instance, a controller of the therapy delivery system 50 can cease delivery of therapy signals to the target location when the detected amount or volume of nasal secretions has reached a normal or healthy level.

Although the ultimate goal of eradicating CRS remains, symptom reduction is a major goal in subjects with CRS. Advantageously, the present disclosure provides methods for assisting with, or replacing, current standard treatments in subjects with CRS whose symptoms are recalcitrant to such standard treatments. By providing both open- and closed-loop therapy delivery systems, the present disclosure permits both on-demand or continuous monitoring and treatment of CRS symptoms, while also delivering therapy on an as-needed basis to improve patients' quality of life.

From the above description of the invention, those skilled in the art will perceive improvements, changes and modifications. For example, present disclosure may be implemented to apply a therapy signal (or therapy signals) to a nerve structure or nervous tissue (e.g., the SPG 20, sphenopalatine nerves 32, and/or vidian nerve 28) on either or both sides of a subject's head. Alternatively, it will be appreciated that the therapy delivery system can be configured to deliver transcutaneous therapy (e.g., electric current) using, for example, magnetic wave therapy as disclosed in U.S. Provisional Patent Application Ser. No. 61/778,521, filed Mar. 13, 2013, the entirety of which is hereby incorporated by reference. Such improvements, changes, and modifications are within the skill of the art and are intended to be covered by the appended claims.

The following is claimed:

1. A method for treating chronic rhinosinusitis (CRS) in a subject, said method comprising the steps of:
implanting a therapy delivery system in the subject so that at least one therapy delivery component of the system is positioned substantially adjacent a target location where modulation of the autonomic nervous system (ANS) is effective to treat CRS, the therapy delivery component including at least one electrode configured to deliver electric current to the target location; and
delivering electric current to the at least one electrode to effect a change in the ANS.

2. The method of claim 1, wherein the target location is a nerve structure associated with the pterygopalatine fossa (PPF).

3. The method of claim 2, wherein the nerve structure includes at least one of a sphenopalatine ganglion (SPG), a vidian nerve, or a sphenopalatine nerve.

4. The method of claim 1, wherein modulation of the ANS is effective to alter at least one of mucosal blood flow, sinusoidal filling, mucosal thickness, and mucosal secretion.

5. The method of claim 1, wherein the change in the ANS is induced by at least one pharmacological agent associated with the therapy delivery system.

6. The method of claim 1, wherein the change in the ANS is induced by at least one biological agent associated with the therapy delivery system.

7. The method of claim 1, wherein said step of implanting the therapy delivery system includes positioning the therapy delivery component substantially adjacent a target location where modulation of the parasympathetic nervous system (PNS) is effective to treat CRS.

8. The method of claim 1, wherein said step of implanting the therapy delivery system includes positioning the therapy delivery component substantially adjacent a target location where modulation of the sympathetic nervous system (SNS) is effective to treat CRS.

9. The method of claim 1, wherein said step of implanting the therapy delivery system includes positioning the therapy delivery component substantially adjacent a target location where modulation of the PNS and SNS is effective to treat CRS.

10. The method of claim 7, wherein modulation of the PNS comprises substantially blocking or reducing parasympathetic activity.

11. The method of claim 10, wherein substantially blocking or reducing parasympathetic activity decreases at least one of swelling of nasal mucosa and mucosal secretion.

12. The method of claim 8, wherein modulation of the SNS comprises substantially activating or increasing sympathetic activity.

13. The method of claim 12, wherein substantially activating or increasing sympathetic activity decreases at least one of mucosal blood flow, sinusoidal filling, and mucosal thickness.

14. The method of claim 2, wherein delivery of electric current to the at least one electrode modulates afferent neuronal activity.

15. The method of claim 2, wherein delivery of electric current to the at least one electrode modulates efferent neuronal activity.

16. A method for treating CRS in a subject, said method comprising the steps of:
implanting a closed-loop therapy delivery system in the subject so that at least one therapy delivery component of the system is positioned substantially adjacent a target location where modulation of the ANS is effective to treat CRS, the therapy delivery component including at least one electrode configured to deliver electric current to the target location;
delivering electric current to the at least one electrode to effect a change in the ANS;
sensing, by the therapy delivery system, of at least one physiological parameter associated with CRS; and
activating the therapy delivery component of the system to adjust application of the electric current to the target site in response to the sensed at least one physiological parameter.

17. The method of claim 16, wherein the target location is a nerve structure associated with the PPF.

18. The method of claim 17, wherein the nerve structure includes at least one of a SPG, a vidian nerve, or a sphenopalatine nerve.

19. The method of claim 16, wherein modulation of the ANS is effective to alter at least one of mucosal blood flow, sinusoidal filling, mucosal thickness, and mucosal secretion.

20. The method of claim 16, wherein said step of implanting the closed-loop therapy delivery system includes positioning the therapy delivery component substantially adjacent a target location where modulation of the PNS and/or SNS is effective to treat CRS.

21. The method of claim 16, wherein said sensing step further comprises:
generating a sensor signal based on the sensed at least one physiological parameter; and
activating the therapy delivery system to adjust application of electric current to the target site in response to the sensor signal to treat CRS.
